(19) United States
(12) Patent Application Publication
(10) Pub. No.: US 2021/0269605 A1
(43) Pub. Date: Sep. 2, 2021

(54) DEVELOPING MATERIAL, MEDICAL TUBE AND PREPARATION METHOD THEREFOR

(71) Applicant: ACCUPATH MEDICAL (JIAXING) CO., LTD., Zhejiang (CN)

(72) Inventors: Zhaomin LI, Zhejiang (CN); Minglin QIN, Zhejiang (CN); Guangbin HE, Zhejiang (CN); Zhihua DENG, Zhejiang (CN); Quanquan SUN, Zhejiang (CN); Ruipei LI, Zhejiang (CN); Yifan LIU, Zhejiang (CN); Yiyun QUE, Zhejiang (CN)

(21) Appl. No.: 17/255,902

(22) PCT Filed: Jun. 28, 2019
(86) PCT No.: PCT/CN2019/093724

§ 371 Date: Dec. 23, 2020

(30) Foreign Application Priority Data
Sep. 30, 2018 (CN) 201811155300.1

(51) Int. Cl. C08J 3/22; A61L 29/18; A61L 29/02; A61L 29/12; A61L 29/06; A61L 29/04; C08K 3/30; C08K 3/08; C08K 3/26; C08K 3/22; C08L 23/12; C08L 27/06; C08L 75/04; C08L 77/00; C08L 77/02; A61M 25/01

(52) U.S. Cl. CPC C08J 3/226; C08L 2207/04; A61L 29/02; A61L 29/126; A61L 29/06; A61L 29/041; A61L 29/042; C08K 3/30; C08K 3/08; C08K 3/26; C08K 3/22; C08L 23/12; C08L 27/06; C08L 75/04; C08L 77/00; C08L 77/02; A61M 25/0108; C08K 2201/005; C08J 2377/00; C08J 2327/06; C08J 2323/12; C08J 2375/04; C08K 2003/3045; C08K 2003/0887; C08L 2203/02; A61L 29/18

(57) ABSTRACT

A preparation method comprises following steps: S1: dissolving a coupling agent in an ethanol solution, adding an ultrafine radiopaque agent powder in the ethanol solution, and obtaining a modified ultrafine radiopaque agent powder by agitation, washing and drying, the ultrafine radiopaque agent powder having a particle size of 0.35-0.8 μm; S2: obtaining the radiopaque material by mixing a medical polymeric material with the modified ultrafine radiopaque agent powder. A prepared radiopaque material exhibits not only radiopaque functions but also good mechanical properties of improved elastic modulus, fracture strength and bending modulus, thereby expanding application of medical polymer hollow fibers in high-end medical products for minimally invasive intervention. Particularly, delivery devices applied with prepared radiopaque material have improved pushing and torque performance and are suitable for ultra-smooth guidewires, heart valve prostheses, guiding catheters, and degradable balloons.

### TECHNICAL FIELD

The present disclosure relates to the field of biomedical raw materials and, in particular, to a biomedical polymeric radiopaque material, a medical tube and preparation method thereof.

### BACKGROUND

Minimally invasive intervention is a medical technique considered as one of the most important contributions of medicine to human civilization at the end of the 20th century, which covers various scientific and technological disciplines including, cardiovascular discipline, cerebrovascular discipline, oncology, surgery, gynecology and otolaryngology. Precision tubes are critical materials for minimally invasive intervention and have been monopolized by European and American companies for long due to demanding requirements and great technical difficulties. In the past 20 years, main challenges that have been faced in China's industrialization of precision tubes for use in medical instruments for minimally invasive intervention include shortage of talents, key technology and critical equipment for blend modification of medical polymeric materials, precision extrusion, welding, grinding, braiding, coiling, condensed matter control, and so on.

### SUMMARY

### Technical Problem

Existing medical polymeric materials are generally of the following four categories: fluorine polymers, polyimides, nylons and thermoplastic elastomers. These materials are widely used in instruments for minimally invasive intervention because of a broad range of hardness, excellent physical and mechanical properties, good biocompatibility (certified as USP Class IV) and good machinability. When producing a catheter for minimally invasive interventional therapy, materials with different hardness grades can be selected to produce different catheter sections to obtain catheters with hardness gradients, as medical polymeric materials of different hardness grades have excellent compatibilities and hot weld strengths. When endowed with a radiopaque performance, the application of such catheters can be further expanded to stents, covered stents, occluders, coils, embolic materials, etc. A traditional approach for endowing the minimally invasive interventional therapy instruments with a radiopaque performance is to weld a radiopaque ring onto the instrument. However, the partial welding might lead to stress concentration, which would affect mechanical properties of the instrument itself. In addition, mixing and co-extrusion of radiopaque agent with a thermoplastic elastomer can also be used to obtain the novel radiopaque material suitable for interventional therapy field. Most traditional radiopaque fibers use barium sulfate as the radiopaque agent. However, in order to meet medical radiopaque requirements, barium sulfate must be present at a content of greater than 60% in the radiopaque fibers. Moreover, as barium sulfate is poorly compatible with the polymer matrix, the radiopaque agent is easy to agglomerate, which degrades the material's mechanical properties and is unfavorable to the radiopaque performance. Further, simple mixing and co-extrusion may also cause agglomeration of the radiopaque agent, thereby bringing an adverse impact on the material's radiopaque performance and decreasing material's mechanical properties.

### Technical Solution to Problem

The technical problem to be solved by present disclosure is to provide a radiopaque material, a medical tube and a preparation method thereof, in which the prepared polymeric material exhibits not only radiopaque functions but also good mechanical properties of improved elastic modulus, fracture strength and bending modulus, thereby expanding application of medical polymer hollow fibers in high-end medical products for minimally invasive intervention.

In order to solve the above technical problem, one aspect of present disclosure provides a method for preparing a radiopaque material, comprising the steps of: (S1) dissolving a coupling agent in an ethanol solution, adding an ultrafine radiopaque agent powder in the ethanol solution, and obtaining a modified ultrafine radiopaque agent powder by agitation, washing and drying, wherein the ultrafine radiopaque agent powder has a particle size of 0.35-0.8 μm; and (S2) obtaining the radiopaque material by mixing a medical polymeric material with the modified ultrafine radiopaque agent powder.

Further, in step S2, before mixing the medical polymeric material with the modified ultrafine radiopaque agent powder, each of the medical polymeric material and the modified ultrafine radiopaque agent powder is dried in vacuum at 70-90° C. for 12-24 h.

Further, the medical polymeric material is one or more selected from the group consisting of polyether block amide, polyvinyl chloride, polypropylene, nylon 6, nylon 12 and thermoplastic polyurethane elastomer rubber.

Further, the ultrafine radiopaque agent powder is one or more selected from the group consisting of ultrafine barium sulfate powder, ultrafine tungsten powder, ultrafine bismuth carbonate powder and ultrafine bismuth oxychloride powder.

Further, in step S1, the dissolution of the coupling agent in the ethanol solution and the addition of the ultrafine radiopaque agent powder in the ethanol solution are followed by high-speed agitation at 65-85° C. for 0.5-1 h, washing with ethanol and deionized water successively for 4-5 runs and drying at a constant temperature of 65-85° C. for 1-2 h.

Further, the coupling agent is one or more selected from the group consisting of aminosilane, acryloyloxysilane, isopropyl dioleyl titanate and isopropyl trititanate.

Further, the coupling agent is present in an amount of from 0.5% to 2% with a total amount of the ultrafine radiopaque agent powder taken as being 100 wt % by weight.

Further, prior to step S2, the medical polymeric material is added to a coating solution, agitated, filtered and dried in ambient air to obtain a pre-treated medical polymeric material, wherein the medical polymeric material is a thermoplastic elastomer, and the coating solution is a polyethylene glycol solution.

Further, in step S2, the modified ultrafine radiopaque agent powder is sprinkled over the pre-treated medical polymeric material at a predetermined ratio and a mixture of the medical polymeric material and the ultrafine radiopaque agent powder is obtained through a homogeneous mixing by a high-speed agitator, and wherein the ultrafine radiopaque agent powder is present in an amount of from 40% to 50% with a total amount of the medical polymeric material taken as being 100 wt % by weight.

Further, prior to step S2, a pre-treated ultrafine radiopaque agent powder is obtained by mixing the modified ultrafine radiopaque agent powder, a dispersant and a carrier at a predetermined ratio with a high-speed agitator to form a homogeneous mixture and extruding the mixture with a co-rotating twin-screw extruder at 65-85° C.

Further, the mixture extruded from the co-rotating twin-screw extruder is washed in a water tank at room temperature, and condensed, granulated and dried with forced air to obtain ultrafine radiopaque agent masterbatches.

Further, in step S2, the medical polymeric material and the ultrafine radiopaque agent masterbatches are homogeneously mixed at a predetermined ratio with a high-speed agitator, and wherein the ultrafine radiopaque agent powder is present in an amount of from 40% to 50% with a total amount of the medical polymeric material taken as being 100 wt % by weight.

Further, the dispersant is a low-molecular polyethylene wax or a stearate, and the carrier is a same thermoplastic elastomer as the medical polymeric material or a low-density polyethylene.

Further, in step S2, the radiopaque material with a radiopaque agent uniformly dispersed therein is obtained by mixing the medical polymeric material with the modified ultrafine radiopaque agent powder to form a mixture, extruding the mixture with a co-rotating twin-screw extruder, washing the mixture in a water tank at room temperature, and condensing, granulating and forced air drying the mixture.

Further, in step S2, the medical polymeric material is mixed with the modified ultrafine radiopaque agent powder to form a mixture, and the mixture is extruded with a co-rotating twin-screw extruder having a screw length to diameter ratio of 1:35 to 1:55 at a screw speed of 200-400 rpm, an extrusion temperature of 180-240° C., a discharge end pressure of 3-5 MPa and a vacuum pump pressure of 0.8 MPa.

In order to solve the above technical problem, second aspect of present disclosure also provides a radiopaque material prepared using the method as defined above.

In order to solve the above problem, third aspect of present disclosure provides a medical comprising the above radiopaque material.

### Beneficial Effect

Compared with the prior art, the radiopaque material, medical tube and preparation method thereof provided in present disclosure offer the following benefits: the radiopaque material provided in present disclosure is prepared by mixing an ultrafine radiopaque agent powder with a biomedical polymeric material. Compared with common radiopaque agents, the ultrafine radiopaque agent powder has a larger specific surface area and a better dispersity. The well dispersed ultrafine radiopaque agent powder enables to improve the elastic modulus, fracture strength and bending modulus of the medical polymeric material. Through addition of coupling agent to modify the surface of the ultrafine radiopaque agent powder, the compatibility and adhesion between the medical polymeric material and the ultrafine radiopaque agent powder are able to be enhanced. Moreover, pretreatments of the medical polymeric material or the ultrafine radiopaque agent powder to modify their surface properties before the mixing allows greatly raise dispersion of the radiopaque agent across the medical polymeric material, which results in the prepared medical polymeric radiopaque material having a distinct radiopaque effect without variations in brightness. In addition, there is no need to introduce a radiopaque ring during the preparation process, and the radiopaque treatment to materials can be achieved by eliminating complicated welding procedures directly, thus simplifying the overall process and increasing productivity. Application of the prepared radiopaque materials to delivery devices enables to improve pushing and torque performance of delivery devices and make delivery devices suitable for ultra-smooth guidewires, heart valve prostheses, guiding catheters, degradable balloons and many other products.

### BRIEF DESCRIPTION OF THE DRAWINGS

FIG. 1 shows the radiopaque performance of the radiopaque material prepared by Example 3 of the present disclosure.

FIG. 2 shows the radiopaque performance of a radiopaque material made from a conventional radiopaque agent.

### DETAILED DESCRIPTION

The present disclosure will be described in greater detail by way of specific examples, which, however, should not be understood as limitations to present disclosure in any sense.

### Example 1

First of all, each of the medical polymeric material and the radiopaque agent in the form of an ultrafine powder is vacuum-dried at 70-90° C. for 12-24 h. Preferably, the medical polymeric material is one or more selected from the group consisting of polyether block amide (PEBA), polyvinyl chloride (PVC), polypropylene (PP), nylon 6, nylon 12 and thermoplastic polyurethane elastomer rubber (TPU). Preferably, the ultrafine radiopaque agent powder is one or more selected from the group consisting of ultrafine barium sulfate powder, ultrafine tungsten powder, ultrafine bismuth carbonate powder and ultrafine bismuth oxychloride powder. The ultrafine radiopaque agent powder has a particle size of 0.35-0.8 μm.

Subsequently, a coupling agent was dissolved in an ethanol solution, and the dried ultrafine radiopaque agent powder was added thereto. The coupling agent accounts for 0.5-2% by weight of the ultrafine radiopaque agent powder. The system was then agitated at a high speed at 65-85° C. for 0.5-1 h to allow a reaction to take place, and a product resulting from the reaction was washed 4-5 times successively, each time using both ethanol and deionized water. A modified ultrafine radiopaque agent powder was obtained after the product was dried at a constant temperature of 65-85° C. for 1-2 h. The coupling agent is preferably one or more selected from the group consisting of aminosilane, acryloyloxysilane, isopropyl dioleic(dioctylphosphate) titanate and isopropyl tri(dioctylphosphate) titanate.

Afterwards, the modified ultrafine radiopaque agent powder is homogeneously mixed with a dispersant and a carrier at a predetermined ratio with a high-speed agitator at 65-85° C., and the mixture was extruded with a co-rotating twin-screw extruder. The dispersant is preferably a low-molecular polyethylene wax with a molecular weight of about 1,000-2,000 Mw or a stearate such as calcium stearate, zinc stearate, lead stearate, barium stearate or the like. The carrier was the same thermoplastic elastomer as the medical polymeric material or the low-density polyethylene, preferably the same resin as the product being fabricated, more preferably a low-density polyethylene with a density in the range of 0.918-0.935 g/cm³.

Finally, the medical polymeric material was homogeneously mixed with the pre-treated ultrafine radiopaque agent powder using a high-speed agitator at a mass ratio of 1:0.4-0.5, and the mixture was extruded with a co-rotating twin-screw extruder, in which temperatures for different sections of extruder is respectively set as 210° C., 220° C., 230° C. and 240° C., a screw length to diameter ratio being 1:35, a screw speed being 200 rpm, a pressure at the discharge end being 3 MPa, a vacuum pump pressure being 0.8 MPa. The resulting extruded products are washed in water contained in a water tank at room temperature and condensed, granulated and dried with forced air, and the radiopaque material was obtained.

### Example 2

First of all, each of the medical polymeric material and the radiopaque agent in the form of an ultrafine powder is vacuum-dried at 70-90° C. for 12-24 h. selected from the group consisting of polyether block amide (PEBA), polyvinyl chloride (PVC), polypropylene (PP), nylon 6, nylon 12 and thermoplastic polyurethane elastomer rubber (TPU). Preferably, the ultrafine radiopaque agent powder is one or more selected from the group consisting of ultrafine barium sulfate powder, ultrafine tungsten powder, ultrafine bismuth carbonate powder and ultrafine bismuth oxychloride powder. The ultrafine radiopaque agent powder has a particle size of 0.35-0.8 μm.

Subsequently, a coupling agent was dissolved in an ethanol solution, and the dried ultrafine radiopaque agent powder was added thereto. The coupling agent accounts for 0.5-2% by weight of the ultrafine radiopaque agent powder. The system was then agitated at a high speed at 65-85° C. for 0.5-1 h to allow a reaction to take place, and a product resulting from the reaction was washed 4-5 times successively, each time using both ethanol and deionized water. A modified ultrafine radiopaque agent powder was obtained after the product was dried at a constant temperature of 65-85° C. for 1-2 h. The coupling agent is preferably one or more selected from the group consisting of aminosilane, acryloyloxysilane, isopropyl dioleic(dioctylphosphate) titanate and isopropyl tri(dioctylphosphate) titanate.

Next, the dried medical polymeric material was added to a coating solution, which was preferably a polyethylene glycol solution. After blending and stirring, filtration and drying in the ambient air, a pre-treated medical polymeric material was thus obtained.

Finally, the pre-treated medical polymeric material was homogeneously mixed with the modified ultrafine radiopaque agent powder using a high-speed agitator at a mass ratio of 1:0.4-0.5, and the mixture was extruded with a co-rotating twin-screw extruder, in which temperatures for different sections of extruder is respectively set as 210° C., 220° C., 230° C. and 240° C., a screw length to diameter ratio being 1:35, a screw speed being 200 rpm, a pressure at the discharge end being 3 MPa, a vacuum pump pressure being 0.8 MPa. The resulting extruded products are washed in water contained in a water tank at room temperature and condensed, granulated and dried with forced air, and the radiopaque material was obtained.

### Example 3

In this example, the ultrafine barium sulfate powder is used to prepared the radiopaque material.

At first, each of the ultrafine barium sulfate powder with a particle size of 0.5 μm and the polyether block amide (PEBA) copolymer is vacuum-dried at 90° C. for 16 h. Next, a coupling agent aminosilane was dissolved in an ethanol solution, and the dried ultrafine barium sulfate powder was added thereto. The aminosilane was added at an amount equal to 2% by weight of the ultrafine barium sulfate powder weight. The system was then agitated at a high speed at 85° C. for 45 min to allow a reaction to take place, and a product resulting from the reaction was washed 4 times successively, each time using both ethanol and deionized water. A modified ultrafine barium sulfate powder was obtained after the product was dried at a constant temperature of 85° C. for 2 h. Subsequently, the modified ultrafine barium sulfate powder was homogeneously mixed with a low-molecular polyethylene wax and a carrier at a predetermined ratio with a high-speed agitator at 85° C., and the mixture was extruded with a co-rotating twin-screw extruder. The extruded mixtures were washed in water contained in a water tank at room temperature, condensed, granulated and dried with forced air, and pre-treated radiopaque masterbatches of the ultrafine barium sulfate powder are obtained.

Finally, the PEBA copolymer was homogeneously mixed with the pre-treated radiopaque masterbatches of the ultrafine barium sulfate powder at a mass ratio of 1:0.5 using a high-speed agitator, and the mixture was extruded with a co-rotating twin-screw extruder, in which temperatures for different sections of extruder is respectively set as 210° C., 220° C., 230° C. and 240° C., a screw length to diameter ratio being 1:35, a screw speed being 200 rpm, a pressure at the discharge end being 3 MPa, a vacuum pump pressure being 0.8 MPa. The resulting extruded products are washed in water contained in a water tank at room temperature and condensed, granulated and dried with forced air, and the radiopaque material was obtained.

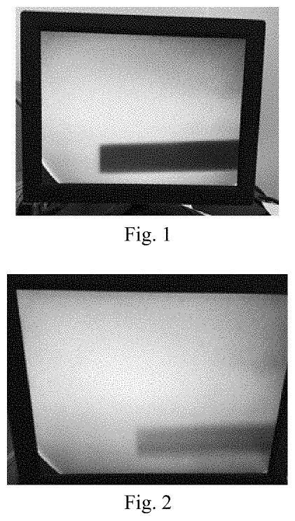

FIG. 1 shows the radiopaque performance of the radiopaque material prepared this example, while FIG. 2 shows the radiopaque performance of a radiopaque material made from a conventional radiopaque agent. As can be seen from FIGS. 1 and 2, the radiopaque material obtained in this example has a sufficient radiopaque performance and demonstrates a sharp outline under the exposure of X-ray radiations. In contrast, the radiopaque performance of the radiopaque material made from the conventional radiopaque agent is insufficient and exhibits a blurred outline under the exposure of X-ray radiations.

In summary, the radiopaque material of the present disclosure is prepared by mixing the ultrafine radiopaque agent powder with a biomedical polymeric material. Compared with powder radiopaque agents, the ultrafine radiopaque agent powder has a larger specific surface area and a better dispersity. The well dispersed ultrafine radiopaque agent powder enables to improve the elastic modulus, fracture strength and bending modulus of the medical polymeric material. Through addition of coupling agent to modify the surface of the ultrafine radiopaque agent powder, the compatibility and adhesion between the medical polymeric material and the ultrafine radiopaque agent powder are able to be enhanced. Moreover, pretreatments of the medical polymeric material or the ultrafine radiopaque agent powder to modify their surface properties before the mixing allows greatly raise dispersion of the radiopaque agent across the medical polymeric material, which results in the prepared medical polymeric radiopaque material having a distinct radiopaque effect without variations in brightness. In addition, there is no need to introduce a radiopaque ring during the preparation process, and the radiopaque treatment to materials can be achieved by eliminating complicated welding procedures directly, thus simplifying the overall process and increasing productivity. Application of the prepared radiopaque materials to delivery devices enables to improve pushing and torque performance of delivery devices and make delivery devices suitable for ultra-smooth guidewires, heart valve prostheses, guiding catheters, degradable balloons and many other products.

Although the present disclosure has been disclosed above by way of preferred embodiments, these embodiments are not intended to limit the present disclosure in any sense. Any and all changes and modifications made by those skilled in the art without departing from the spirit and scope of the present disclosure are intended to be embraced within the protection scope of present disclosure.

1. A method for preparing a radiopaque material, comprising steps of:
(S1) dissolving a coupling agent in an ethanol solution, adding an ultrafine radiopaque agent powder in the ethanol solution, and obtaining a modified ultrafine radiopaque agent powder by agitation, washing and drying, wherein the ultrafine radiopaque agent powder has a particle size of 0.35-0.8 μm; and
(S2) obtaining the radiopaque material by mixing a medical polymeric material with the modified ultrafine radiopaque agent powder.

2. The method of claim 1, wherein in step S2, before mixing the medical polymeric material with the modified ultrafine radiopaque agent powder, each of the medical polymeric material and the modified ultrafine radiopaque agent powder is dried in vacuum at 70-90° C. for 12-24 h.

3. The method of claim 1, wherein the medical polymeric material is one or more selected from the group consisting of polyether block amide, polyvinyl chloride, polypropylene, nylon 6, nylon 12 and thermoplastic polyurethane elastomer rubber.

4. The method of claim 1, wherein the ultrafine radiopaque agent powder is one or more selected from the group consisting of ultrafine barium sulfate powder, ultrafine tungsten powder, ultrafine bismuth carbonate powder and ultrafine bismuth oxychloride powder.

5. The method of claim 1, wherein in step S1, the dissolution of the coupling agent in the ethanol solution and the addition of the ultrafine radiopaque agent powder in the ethanol solution are followed by high-speed agitation at 65-85° C. for 0.5-1 h, washing with ethanol and deionized water successively for 4-5 times and drying at a constant temperature of 65-85° C. for 1-2 h.

6. The method of claim 1, wherein the coupling agent is one or more selected from the group consisting of aminosilane, acryloyloxysilane, isopropyl dioleyl titanate and isopropyl trititanate.

7. The method of claim 1, wherein the coupling agent is present in an amount of from 0.5% to 2% with a total amount of the ultrafine radiopaque agent powder taken as being 100 wt % by weight.

8. The method of claim 1, wherein prior to step S2, the medical polymeric material is added to a coating solution, agitated, filtered and dried in ambient air to obtain a pre-treated medical polymeric material, wherein the medical polymeric material is a thermoplastic elastomer, and the coating solution is a polyethylene glycol solution.

9. The method of claim 1, wherein in step S2, the modified ultrafine radiopaque agent powder is sprinkled over the pre-treated medical polymeric material at a predetermined ratio and a mixture of the medical polymeric material and the ultrafine radiopaque agent powder is obtained through a homogeneous mixing by a high-speed agitator, and wherein the ultrafine radiopaque agent powder is present in an amount of from 40% to 50% with a total amount of the medical polymeric material taken as being 100 wt % by weight.

10. The method of claim 1, wherein prior to step S2, a pre-treated ultrafine radiopaque agent powder is obtained by mixing the modified ultrafine radiopaque agent powder, a dispersant and a carrier at a predetermined ratio with a high-speed agitator to form a homogeneous mixture and extruding the mixture with a co-rotating twin-screw extruder at 65-85° C.

11. The method of claim 10, wherein the mixture extruded from the co-rotating twin-screw extruder is washed in a water tank at room temperature, and condensed, granulated and dried with forced air to obtain ultrafine radiopaque agent masterbatches.

12. The method of claim 10, wherein in step S2, the medical polymeric material and the ultrafine radiopaque agent masterbatches are homogeneously mixed at a predetermined ratio with a high-speed agitator, and wherein the ultrafine radiopaque agent powder is present in an amount of from 40% to 50% with a total amount of the medical polymeric material taken as being 100 wt % by weight.

13. The method of claim 10, wherein the dispersant is a low-molecular polyethylene wax or a stearate, and wherein the carrier is a same thermoplastic elastomer as the medical polymeric material or a low-density polyethylene.

14. The method of claim 1, wherein in step S2, the radiopaque material with a radiopaque agent uniformly dispersed therein is obtained by mixing the medical polymeric material with the modified ultrafine radiopaque agent powder to form a mixture, extruding the mixture with a co-rotating twin-screw extruder, washing the mixture in a water tank at room temperature, and condensing, granulating and forced air drying the mixture.

15. The method of claim 1, wherein in step S2, the medical polymeric material is mixed with the modified ultrafine radiopaque agent powder to form a mixture, and the mixture is extruded with a co-rotating twin-screw extruder having a screw length to diameter ratio of 1:35 to 1:55 at a screw speed of 200-400 rpm, an extrusion temperature of 180-240° C., a discharge end pressure of 3-5 MPa and a vacuum pump pressure of 0.8 MPa.

16. A radiopaque material prepared using the method of claim 1.

17. A medical tube, comprising the radiopaque material of claim 16.
